(19) United States
(12) Patent Application Publication
(10) Pub. No.: US 2023/0194547 A1
(43) Pub. Date: Jun. 22, 2023

(54) LARGE MOLECULE UNSPECIFIC CLEARANCE ASSAY

(71) Applicant: Hoffmann-La Roche Inc., Little Falls, NJ (US)

(72) Inventors: Anna-Lena BOLENDER, Penzberg (DE); Martina DUSCHMALE, Bettingen (CH); Lisa EBELER, Munich (DE); Johannes FRAIDLING, Wessobrunn (DE); Thomas KRAFT, Munich (DE)

(73) Assignee: Hoffmann-La Roche Inc., Little Falls, NJ (US)

(21) Appl. No.: 18/045,037

(22) Filed: Oct. 7, 2022

Related U.S. Application Data
(63) Continuation of application No. PCT/EP2021/058839, filed on Apr. 6, 2021.

(30) Foreign Application Priority Data
Apr. 8, 2020 (EP) 20168671.4

(51) Int. Cl. G01N 33/68; G01N 33/58; G01N 33/50

(52) U.S. Cl. CPC G01N 33/6854; G01N 33/582; G01N 33/5061; G01N 2333/70535

(57) ABSTRACT

Herein is reported a method for determining non-specific clearance of an antibody comprising the steps of incubating the antibody, which is conjugated to a pH-sensitive fluorescent dye, with primary human endothelial cells, and determining the fluorescence intensity of the primary human endothelial cells, whereby an increase of the fluorescence intensity of the primary human endothelial cells above background level is indicative for non-specific clearance of the antibody.

### CROSS REFERENCE TO RELATED APPLICATIONS

This application is a Continuation of International Application No. PCT/EP2021/058839 filed Apr. 6, 2021, claiming priority to EP Application No, 20168671.4 filed Apr. 8, 2020, which are incorporated herein by reference in their entirety.

### SEQUENCE LISTING

This application contains a Sequence Listing which has been submitted electronically in XML format and is hereby incorporated by reference in its entirety. Said XML copy, created on Oct. 5, 2022, is named Sequence_listing and is 8,182 bytes in size.

Herein is reported a novel method for the estimation of the clearance of therapeutic proteins in humans using a novel human primary cell-based in vitro assay. This Large Molecule Unspecific Clearance Assay (LUCA) provides for an in vitro-based method to assess and predict major PK properties for therapeutic proteins.

### BACKGROUND OF THE INVENTION

Human immunoglobulins of the class G (IgGs) contain two antigen binding (Fab) regions that convey specificity for the target antigen and a constant region (Fc-region) that is responsible for interactions with Fc receptors (see e.g. Edelman, G. M., Scand. J. Immunol. 34 (1991) 1-22; Reff, M. E. and Heard, C., Crit. Rev. Oncol. Hematol. 40 (2001) 25-35). Human IgGs of subclasses IgG1, IgG2 and IgG4 have an average serum half-life of 21 days, which is longer than that of any other known serum protein (see, e.g., Waldmann, T. A. and Strober, W., Prog. Allergy 13 (1969) 1-110). This long half-life is predominantly mediated by the interaction between the Fc-region and the neonatal Fc receptor (FcRn) (see, e.g. Ghetie, V. and Ward, E. S., Annu. Rev. Immunol. 18 (2000) 739-766; Chaudhury, C., et al., J. Exp. Med. 197 (2003) 315-322.). This is one of the reasons, why IgGs or Fc-containing fusion proteins are used as a widespread class of therapeutics.

The neonatal Fc receptor FcRn is a membrane-associated receptor involved in both IgG and albumin homeostasis, in maternal IgG transport across the placenta and in antigen-IgG immune complex phagocytosis (see, e.g., Brambell, F. W., et al., Nature 203 (1964) 1352-1354; Ropeenian, D. C., et al., J. Immunol. 170 (2003) 3528-3533). Human FcRn is a heterodimer consisting of the glycosylated class I major histocompatibility complex-like protein (α-FcRn) and a (β2 microglobulin ((β2m) subunit (see, e.g., Kuo, T. T., et al., J. Clin. Immunol. 30 (2010) 777-789). FcRn binds to a site in the CH2-CH3 region of the Fc-region (see, e.g., Ropeenian, D. C. and Akilesh, S., Nat. Rev. Immunol. 7 (2007) 715-725; Martin, W. L., et al., Mol. Cell 7 (2001) 867-877; Goebl, N. A., et al., Mol. Biol. Cell 19 (2008) 5490-5505; Kim, J. K., et al., Eur. J. Immunol. 24 (1994) 542-548.) and two FcRn molecules can bind to the Fc-region simultaneously (see, e.g., Sanchez, L. M., et al., Biochemistry 38 (1999) 9471-9476; Huber, A. H., et al., J. Mol. Biol. 230 (1993) 1077-1083.). The affinity between the FcRn and the Fc-region is pH dependent, showing nanomolar affinity at endosomal pH of 5-6 and rather weak binding at a physiological pH of 7.4 (see, e.g., Goebl, N. A., et al., Mol. Biol. Cell 19 (2008) 5490-5505; Ober, R. J., et al., Proc. Natl. Acad. Sci. USA 101 (2004) 11076-11081; Ober, R. J., et al., J. Immunol. 172 (2004) 2021-2029). The underlying mechanism conveying long half-life to IgGs can be explained by three fundamental steps. First, IgGs are subject to unspecific pinocytosis by various cell types (see, e.g., Akilesh, S., et al., J. Immunol. 179 (2007) 4580-4588; Montoyo, H. P., et al., Proc. Natl. Acad. Sci. USA 106 (2009) 2788-2793.). Second, IgGs encounter and bind FcRn in the acidic endosome at a pH of 5-6, thereby protecting IgGs from lysosomal degradation (see, e.g., Ropeenian, D. C. and Akilesh, S., Nat. Rev. Immunol. 7 (2007) 715-725; Rodewald, R., J. Cell Biol. 71 (1976) 666-669). Finally, IgGs are released in the extracellular space at physiological pH of 7.4 (see, e.g., Ghetie, V. and Ward, E. S., Annu. Rev. Immunol. 18 (2000) 739-766). This strict pH-dependent bind-and-release mechanism is critical for IgG recycling and any deviation of the binding characteristics at different pH values may strongly influence circulation half-life of IgGs (see, e.g., Vaccaro, C., et al., Nat. Biotechnol. 23 (2005) 1283-1288).

Eigenmann, M. J., et al. outlined that cellular uptake of antibodies is thought to occur mostly in endothelial and hematopoietic cells. Once antibodies are taken up into the endosome, they can be protected from degradation by binding to the neonatal Fc receptor (FcRn). This receptor binds antibodies in a pH-dependent manner with higher affinity at pH 6 in the endosome than at the physiologic pH of »7.4 in plasma. Therefore, antibodies that are bound to FcRn in the endosome are released at neutral pH into the plasma, allowing recirculation of the antibody rather than lysosomal degradation (MABS 9 (2017) 1007-1015).

Grevys, A., et al., reported a human endothelial cell-based recycling assay for screening of FcRn targeted molecules (Nat. Commun. 9 (2018) 621). A homogeneous plate based antibody internalization assay using pH sensor fluorescent dye was reported by Nath, N., et al. (J. Immunol. Meth. 431 (2016) 11-21).

In WO 2013/134686 fluorescent sensor agents and methods of use and manufacture thereof are provided. In particular, sensor agents are provided that exhibit a detectable change in fluorescence (e.g., fluorescence intensity) upon alteration of the pH of the surrounding environment (e.g., upon moving from one pH environment to another).

Based on the major biological contributors of non-specific clearance of therapeutic antibodies in patients, namely unspecific uptake via pinocytosis and FcRn-mediated recycling, there is a need for in vitro methods for predicting in vivo clearance, i.e. half-life.

### SUMMARY OF THE INVENTION

Herein is reported a method for determining the level of non-specific clearance of a therapeutic protein, especially an antibody, by pinocytosis and lysosomal degradation.

The current invention is based, at least in part, on the finding that uptake of an antibody into primary human endothelial cells in vitro can be used as surrogate for estimating non-specific clearance of said antibody in vivo, especially in mice, cynomolgus monkey and humans.

The current invention is based, at least in part, on the finding that only primary human endothelial cells can be used for determining the in vivo clearance from an in vitro experiment because non-primary endothelial cells do not show the same correlation and, thus, are not suitable for this purpose. With said non-primary endothelial cells no differentiation between different antibodies can be achieved.

The current invention is based, at least in part, on the finding that the uptake of an antibody by pinocytosis and its routing to the lysosomal compartment of a primary endothelial cell accounts for the largest contribution and shows a good correlation to the primary endothelial cell's fluorescence.

Thus, the current invention comprises a method for determining or estimating the non-specific (i.e. non-target mediated) clearance (rate) of an antibody comprising the following steps:
a) incubating the antibody, which is conjugated to a pH-sensitive fluorescent dye, with primary human endothelial cells (for a defined time), and
b) determining the (intracellular) fluorescence intensity of the primary human endothelial cells obtained in step a) (after the defined incubation time),
whereby by an increase of the (intracellular) fluorescence intensity of the primary human endothelial cells above background level (i.e. the (intracellular) fluorescence of the primary human endothelial cells that had not been incubated with the antibody) determined in step b) the presence of non-specific clearance of the antibody is determined (i.e. is indicative for non-specific clearance of the antibody).

In certain embodiments, the method further comprises the following step:
determining the (intracellular) fluorescence intensity of the primary human endothelial cells prior to the incubation with the antibody/not incubated with the antibody,
and
by an increase of the (intracellular) fluorescence intensity of the primary human endothelial cells determined in step b) above the (intracellular) fluorescence intensity determined for the primary human endothelial cells in the absence of the antibody the presence of non-specific clearance of the antibody is determined (i.e. is indicative for non-specific clearance of the antibody).

Further, the current invention comprises a method for selecting one or more antibodies with low relative non-specific (non-target mediated) clearance (rates) from a multitude of antibodies comprising the following steps:
a) separately incubating for the same defined time each antibody of the multitude of antibodies with primary human endothelial cells, and determining the (intracellular) fluorescence intensity (change) of the primary human endothelial cells thereafter (i.e. determining the change in fluorescence intensity), whereby each antibody is conjugated to the same pH-sensitive fluorescent dye,
b) selecting one or more antibodies from the multitude of antibodies that result in the lowest (intracellular) fluorescence intensity (change) of the primary human endothelial cells after the incubation,
thereby selecting one or more antibodies with low relative non-specific (non-target mediated) clearance (rates).

Further, the current invention comprises a method for ranking antibodies of a multitude of antibodies based on their non-specific (non-target mediated) clearance (rates) comprising the following steps:
a) separately incubating for the same defined time each antibody of the multitude of antibodies with primary human endothelial cells, and determining the (intracellular) fluorescence intensity (change) of the primary human endothelial cells thereafter, whereby each antibody is conjugated to the same pH-sensitive fluorescent dye,
b) ordering the antibodies based on the (intracellular) fluorescence intensity (change) from low to high or high to low, thereby ranking the antibodies based on their non-specific (non-target mediated) clearance (rates).

Further, the current invention comprises a method for estimating or determining the (relative) in vivo clearance rate of an antibody in human or cynomolgus or mouse comprising the following steps:
a) incubating the antibody, which is conjugated to a pH-sensitive fluorescent dye, with primary human endothelial cells for a defined time, and determining the (intracellular) fluorescence intensity (change) of the primary human endothelial cells thereafter,
b) incubating for the same defined time as in a) at least a first reference antibody, for which the human or cynomolgus or murine clearance rate is known and which is conjugated to (in one preferred embodiment the same as in a)) pH-sensitive fluorescent dye, with primary human endothelial cells, and determining the (intracellular) fluorescence intensity (change) of the primary human endothelial cells thereafter,
whereby the (relative) in vivo clearance rate of the antibody in human or cynomolgus or mouse is estimated or determined to be the clearance rate of the first reference antibody in human or cynomolgus or mouse multiplied by the ratio of the (intracellular) fluorescence intensity (change) determined in a) to the (intracellular) fluorescence intensity (change) determined in b).

In certain embodiments, step b) is
b) i) (separately) incubating for the same defined time as in a) each member of a multitude of reference antibodies (i.e. at least two), for which the human or cynomolgus or murine clearance rates are known and which are conjugated to a (in one preferred embodiment the same as in a)) pH-sensitive fluorescent dye, with primary human endothelial cells,
   ii) determining for each of the reference antibodies the (intracellular) fluorescence intensity (change) of the primary human endothelial cells thereafter, and
   iii) calculating for the values obtained in ii) the best fit straight line of the formula y=a*x+b whereby y is the clearance rate in ml/day/kg and x corresponds to the fluorescence intensity (change).

In one embodiment of all aspects and embodiments, the (intracellular) fluorescence intensity (change) is the geometric mean (intracellular) fluorescence intensity (change).

In one embodiment of all aspects and embodiment the (intracellular) fluorescence intensity (change) of the respective antibody in question is a relative normalized (intracellular) fluorescence intensity (change) rate obtained in an additional step c) comprising
1) determining the (geometric mean) (intracellular) fluorescence intensity after two or more defined incubation times for the antibody in question and at least two reference antibodies, whereby in a preferred embodiment the determining is at least for two time points after an incubation time of 2 hours and of 4 hours;
2) subtracting separately from each of the (geometric mean) (intracellular) fluorescence intensities determined in 1) for each of the antibody in question and the reference antibodies the (geometric mean) (intracellular) fluorescence intensity of the primary human endothelial cells (incubated for the same time but without an antibody being present) thereby obtaining corrected (geometric mean) (intracellular) fluorescence intensities;
3) dividing separately the corrected (geometric mean) (intracellular) fluorescence intensities obtained in 2) of the antibody in question and the reference antibodies by the number of fluorescent dye molecules present in the respective antibody to obtain a normalized (geometric mean) (intracellular) fluorescence intensity (e.g. of the at least two reference antibodies or the antibody is question);
4) determining the slope of the best fit straight line (i.e. a linear regression curve y=s*x+b with y=normalized (geometric mean) (intracellular) fluorescence intensity, s=slope, x=time and b=y-axis intersection) for each of the antibody in question and the reference antibodies based on the group of values consisting of the normalized (geometric mean) (intracellular) fluorescence intensities for the at least two different incubation times for the (i.e. each individual) antibody as calculated in 3) and including the point of origin;
5) normalizing the slope of the best-fit straight line of the antibody in question as follows:

$$normalized slope ( antibody in question ) = \frac{s l ope ( antibody in question ) - s l ope ( reference antibody with slower clearance )}{slope ( reference antibody with faster clearance ) - slope ( reference antibody with slower clearance )}$$

In one embodiment of all aspects and embodiment the incubated primary human endothelial cells are washed prior to the determination of the (intracellular) fluorescence (to remove non-specifically/outer cell surface-bound and unbound antibody).

In one embodiment of all aspects and embodiments the dye has a fluorescence intensity change between a physiological pH of about 7 and an acidic pH in the range of pH 4 to 5 of about 10-fold, preferably about 25-fold and most preferably of about 50-fold. In certain embodiments, the dye has the Formula I/is pHAb of Formula I.

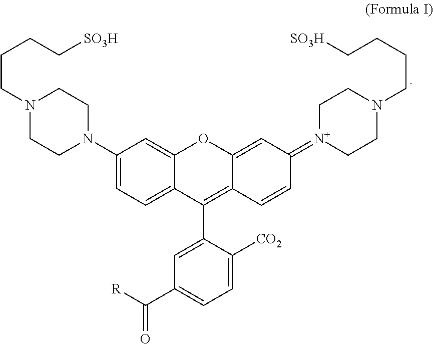

The conjugation to the antibody or the linker, if resent, is at residue R of Formula I.

In one embodiment of all aspects and embodiments, the dye is conjugated to the antibody at amino acid residue 297 in the Fc-region (numbering according to Kabat).

In one embodiment of all aspects and embodiments, the dye is conjugated to the antibody by click-chemistry.

In one embodiment of all aspects and embodiments, the dye is conjugated to the antibody directly or via a linker. In certain embodiments, the linker is Sulfo DBCO-PEG4-Amine of Formula II.

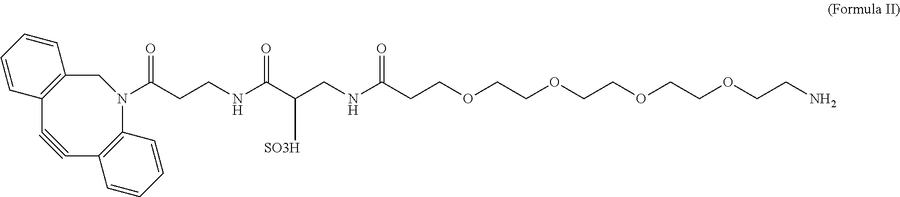 [SMILES: NCCOCCOCCOCCOCCC(=O)NCC(C(=O)NCCC(=O)N1Cc2ccccc2C#Cc2ccccc21)S(=O)(=O)O]

The conjugation to the antibody is at the free amino group of Formula II.

In one embodiment of all aspects and embodiments, the dye is conjugated to a linker and the linker is conjugated to the antibody and this conjugate has a structure of Formula III.

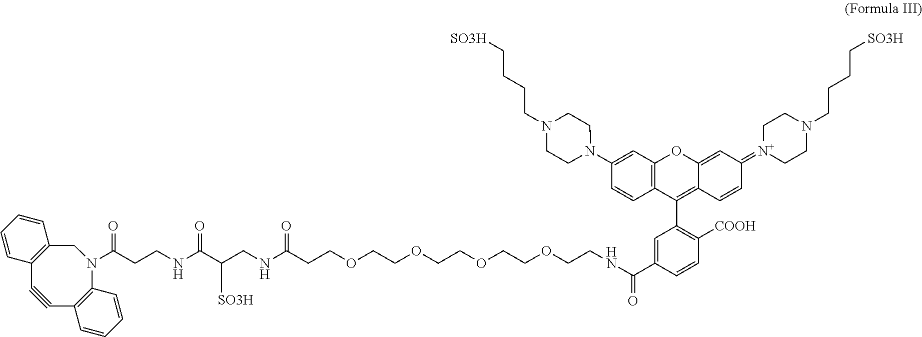 [SMILES: O=C(CCOCCOCCOCCOCCNC(=O)c1ccc(C(=O)O)c(-c2c3ccc(=[N+]4CCN(CCCCS(=O)(=O)O)CC4)cc-3oc3cc(N4CCN(CCCCS(=O)(=O)O)CC4)ccc23)c1)NCC(C(=O)NCCC(=O)N1Cc2ccccc2C#Cc2ccccc21)S(=O)(=O)O]

In one embodiment of all aspects and embodiments, the dye is conjugated to the antibody by chemical crosslinking.

In one embodiment of all aspects and embodiments, the fluorescence is determined by FACS by determining the shift of the fluorescence maximum.

In one embodiment of all aspects and embodiments, the fluorescence is the geometrical mean fluorescence intensity determined by FACS.

In one embodiment of all aspects and embodiments, the primary human endothelial cells are primary human liver endothelial cells.

In one embodiment of all aspects and embodiments, the determining is after an incubation of at least 0.5 hours, i.e. the defined time is at least 0.5 hours.

In one embodiment of all aspects and embodiments, the determining is after an incubating for up to 24 hours, i.e. the defined time is up to 24 hours. In certain embodiments, the determining is after an incubating for up to 16 hours. In one preferred embodiment the determining is after an incubating for up to 4 hours, i.e. the defined time is up to 4 hours. In certain embodiments, the determining is after an incubating for 2 hours or/and 4 hours, i.e. the defined time is 2 hours or/and 4 hours. In certain embodiments, the determining is after an incubating for 4 to 24 hours, i.e. the defined time is between and including 4 hours to 24 hours. In certain embodiments, the determining is after an incubating for 4 hours or/and 8 hours, i.e. the defined time is 4 hours or/and 8 hours.

In certain embodiments, the determining is directly after the incubating.

In one embodiment of all aspects and embodiment, the antibody has an Fc-region of human origin. In certain embodiments, the Fc-region is of the human IgG1 or IgG2 or IgG4 subclass. In certain embodiments, the Fc-region comprises one or more mutations influencing binding to human FcRn.

In one embodiment of all aspects and embodiments, the antibody is a fusion of an antibody with a further polypeptide. In certain embodiments, the further polypeptide is a scFv, a Fab, a scFab or a non-antibody polypeptide. In certain embodiments, the fusion is at the C-terminus of one of the heavy chains of the antibody.

In one embodiment of all aspects and embodiments, the antibody is a bispecific antibody.

In one embodiment of all aspects and embodiment, the first reference antibody is motavizumab with the mutations M252Y/S254T/T256E, and/or a bispecific antibody in TCB format.

### DETAILED DESCRIPTION OF EMBODIMENTS OF THE INVENTION

The current invention is based, at least in part, on the finding that a cell-based assay using primary human endothelial cells can be used for the in vitro estimation of the in vivo rate of lysosomal degradation of therapeutic antibodies.

By using human primary cells the current inventors have found a significant correlation of the readout of the method according to the invention to non-specific clearance in humans. This has been shown for more than 20 therapeutic antibodies in clinical trial stage or marketed. It has further been found by the current inventors that the method according to the invention is likewise applicable to conventional bispecific, monoclonal antibodies reflecting the Y-shape of wild-type human antibodies as well as to non-conventional bispecific antibodies having different formats than wild-type human antibodies and more than two valences as well as antibody-Fc-region fusions. This provides evidence for the general applicability of the correlation of the read-out of the method according to the current invention and mouse, cynomolgus as well as human clearance.

The method according to the current invention can be used to evaluate the pharmacokinetic (PK) properties of different antibody molecules (differing in format, valency and specificity).

Thus, the method according to the current invention can be used
to support the selection of a suitable clinical lead molecule with respect to PK (pharmacokinetic) properties (clearance and half-life, respectively);
to deselect antibodies from a library that have the PK properties not suitable for a therapeutic application, i.e. that have high clearance or short in vivo half-life, respectively;
to rank the members of a group of antibodies with respect to their PK properties (clearance and half-life, respectively);
to determine the relative in vivo clearance rate for an antibody in question only based on those of reference antibodies (or a number of reference antibodies) with known PK properties, i.e. without the need to do in vivo testing;
to guide antibody engineering of PK properties (either by altering the FcRn affinity of the Fc-region or by engineering charged patches of the Fab (the latter being described in WO 2018/197533);
to determine the need for PK engineering and assessing the outcome of the PK engineering.

Thus, the assay according to the current invention can reduce or even replace animal PK studies.

### I. Definitions

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, Md. (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

The knobs into holes dimerization modules and their use in antibody engineering are described in Carter P.; Ridgway J. B. B.; Presta L. G.: Immunotechnology, Volume 2, Number 1, February 1996, pp. 73-73(1).

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991).

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and II (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In certain embodiments, the term about denotes a range of +/−10% of the thereafter-following numerical value. In certain embodiments, the term about denotes a range of +/−5% of the thereafter-following numerical value.

The term “determine” as used herein encompasses also the terms measure and analyze.

The term “comprising” also includes the term “consisting of”.

The term “antibody” herein is used in a broad sense and encompasses various antibody structures, including but not limited to monoclonal antibodies and multispecific antibodies (e.g. bispecific antibodies, trispecific antibodies) so long as they are full-length antibodies and exhibit the desired antigen- and/or FcRn-binding activity.

A “multispecific antibody” denotes an antibody that has binding specificities for at least two different epitopes on the same antigen or two different antigens. Multispecific antibodies can be prepared as full-length antibodies or antibody fragments (e.g. F(ab′)2 bispecific antibodies) or combinations thereof (e.g. full length antibody plus additional scFv or Fab fragments). Engineered antibodies with two, three or more (e.g. four) functional antigen binding sites have also been reported (see, e.g., US 2002/0004587 A1).

The term “binding (to an antigen)” denotes the binding of an antibody in an in vitro assay. In certain embodiments, binding is determined in a binding assay in which the antibody is bound to a surface and binding of the antigen to the antibody is measured by Surface Plasmon Resonance (SPR). The term “binding” also includes the term “specifically binding”.

The term “buffer substance” denotes a substance that when in solution can level changes of the pH value of the solution e.g. due to the addition or release of acidic or basic substances.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc-fusion polypeptide” denotes a fusion of a binding domain (e.g. an antigen binding domain such as a single chain antibody, or a polypeptide such as a ligand of a receptor) with an antibody Fc-region that exhibits the desired target- and/or protein A and/or FcRn-binding activity.

The term “Fc-region of human origin” denotes the C-terminal region of an immunoglobulin heavy chain of human origin that contains at least a part of the hinge region, the CH2 domain and the CH3 domain. In certain embodiments, a human IgG heavy chain Fc-region extends from Cys226, or from Pro230, to the carboxyl-terminus of the heavy chain. In certain embodiments, the Fc-region has the amino acid sequence of SEQ ID NO: 05. However, the C-terminal lysine (Lys447) of the Fc-region may or may not be present. The Fc-region is composed of two heavy chain Fc-region polypeptides, which can be covalently linked to each other via the hinge region cysteine residues forming inter-chain disulfide bonds.

The term “FcRn” denotes the human neonatal Fc-receptor. FcRn functions to salvage IgG from the lysosomal degradation pathway, resulting in reduced clearance and increased half-life. The FcRn is a heterodimeric protein consisting of two polypeptides: a 50 kDa class I major histocompatibility complex-like protein (α-FcRn) and a 15 kDa β2-microglobulin (β2m). FcRn binds with high affinity to the CH2-CH3 portion of the Fc-region of IgG. The interaction between IgG and FcRn is strictly pH dependent and occurs in a 1:2 stoichiometry, with one IgG binding to two FcRn molecules via its two heavy chains (Huber, A. H., et al., J. Mol. Biol. 230 (1993) 1077-1083). FcRn binding occurs in the endosome at acidic pH (pH<6.5) and IgG is released at the neutral cell surface (pH of about 7.4). The pH-sensitive nature of the interaction facilitates the FcRn-mediated protection of IgGs pinocytosed into cells from intracellular degradation by binding to the receptor within the acidic environment of endosomes. FcRn then facilitates the recycling of IgG to the cell surface and subsequent release into the blood stream upon exposure of the FcRn-IgG complex to the neutral pH environment outside the cell.

The term “FcRn binding portion of an Fc-region” denotes the part of an antibody heavy chain polypeptide that extends approximately from EU position 243 to EU position 261 and approximately from EU position 275 to EU position 293 and approximately from EU position 302 to EU position 319 and approximately from EU position 336 to EU position 348 and approximately from EU position 367 to EU position 393 and EU position 408 and approximately from EU position 424 to EU position 440. In certain embodiments, one or more of the following amino acid residues according to the EU numbering of Kabat are altered F243, P244, P245 P, K246, P247, K248, D249, T250, L251, M252, 1253, S254, R255, T256, P257, E258, V259, T260, C261, F275, N276, W277, Y278, V279, D280, V282, E283, V284, H285, N286, A287, K288, T289, K290, P291, R292, E293, V302, V303, S304, V305, L306, T307, V308, L309, H310, Q311, D312, W313, L314, N315, G316, K317, E318, Y319, 1336, S337, K338, A339, K340, G341, Q342, P343, R344, E345, P346, Q347, V348, C367, V369, F372, Y373, P374, S375, D376, 1377, A378, V379, E380, W381, E382, S383, N384, G385, Q386, P387, E388, N389, Y391, T393, S408, S424, C425, S426, V427, M428, H429, E430, A431, L432, H433, N434, H435, Y436, T437, Q438, K439, and S440 (EU numbering).

The term “full length antibody” denotes an antibody having a structure substantially similar to a native antibody structure. A full length antibody comprises two full length antibody light chains comprising a light chain variable domain and a light chain constant domain and two full length antibody heavy chains comprising a heavy chain variable domain, a first constant domain, a hinge region, a second constant domain and a third constant domain. A full-length antibody may comprise further domains, such as e.g. additional scFv or a scFab conjugated to one or more of the chains of the full-length antibody. These conjugates are also encompassed by the term full-length antibody.

The term “derived from” denotes that an amino acid sequence is derived from a parent amino acid sequence by introducing alterations at at least one position. Thus, a derived amino acid sequence differs from the corresponding parent amino acid sequence at at least one corresponding position (numbering according to Kabat EU index for antibody Fc-regions). In certain embodiments, an amino acid sequence derived from a parent amino acid sequence differs by one to fifteen amino acid residues at corresponding positions. In certain embodiments, an amino acid sequence derived from a parent amino acid sequence differs by one to ten amino acid residues at corresponding positions. In certain embodiments, an amino acid sequence derived from a parent amino acid sequence differs by one to six amino acid residues at corresponding positions. Likewise, a derived amino acid sequence has a high amino acid sequence identity to its parent amino acid sequence. In certain embodiments, an amino acid sequence derived from a parent amino acid sequence has 80% or more amino acid sequence identity. In certain embodiments, an amino acid sequence derived from a parent amino acid sequence has 90% or more amino acid sequence identity. In certain embodiments, an amino acid sequence derived from a parent amino acid sequence has 95% or more amino acid sequence identity.

The term “human Fc-region polypeptide” denotes an amino acid sequence that is identical to a “native” or “wild-type” human Fc-region polypeptide. The term “variant (human) Fc-region polypeptide” denotes an amino acid sequence that derived from a “native” or “wild-type” human Fc-region polypeptide by virtue of at least one “amino acid alteration”. A “human Fc-region” is consisting of two human Fc-region polypeptides. A “variant (human) Fc-region” is consisting of two Fc-region polypeptides, whereby both can be variant (human) Fc-region polypeptides or one is a human Fc-region polypeptide and the other is a variant (human) Fc-region polypeptide.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., the CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “isolated” antibody is one that has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., size exclusion chromatography or ion exchange or reverse phase HPLC). For review of methods for assessment of antibody purity, see, e.g., Flatman, S. et al., J. Chrom. B 848 (2007) 79-87.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject., A pharmaceutically acceptable carrier includes, but is not limited to, a buffer, excipient, stabilizer, or preservative.

The term “recombinant antibody”, as used herein, denotes all antibodies (chimeric, humanized and human) that are prepared, expressed, created or isolated by recombinant means. This includes antibodies isolated from a host cell such as a NSO, HEK, BHK or CHO cell or from an animal (e.g. a mouse) that is transgenic for human immunoglobulin genes or antibodies expressed using a recombinant expression plasmid transfected into a host cell. Such recombinant antibodies have variable and constant regions in a rearranged form. The recombinant antibodies as reported herein can be subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The term “TCB”, as used herein, denotes a T-cell bispecific antibody. Such an antibody can have a format as described, e.g. in WO 2013/026831. Those molecules can simultaneously bind to CD3 (first specificity) on T-cells and to an antigen on a target (e.g. tumor) cell (second specificity) and thereby induce killing of target cells. TCB are trivalent bispecific antibodies consisting of four polypeptides or polypeptide chains: one light chain, which is a full length light chain; a further light chain, which is a domain exchanged full length light chain; one heavy chain, which is a full length heavy chain; and a further heavy chain, which is an extended heavy chain comprising an addition domain exchanged heavy or light chain Fab fragment.

In one preferred embodiment a TCB comprises
a) a first and a second Fab fragment that each specifically bind to a first antigen,
b) one domain exchanged Fab fragment that specifically binds to a second antigen in which the CH1 and the CL domain are exchanged for each other,
c) one Fc-region comprising a first heavy chain Fc-region polypeptide and a second heavy chain Fc-region polypeptide,
wherein the C-terminus of CH1 domain of the first Fab fragment is connected to the N-terminus of one of the heavy chain Fc-region polypeptides and the C-terminus of the CL-domain of the domain exchanged Fab fragment is connected to the N-terminus of the other heavy chain Fc-region polypeptide, and
wherein the C-terminus of the CH1 domain of the second Fab fragment is connected to the N-terminus of the VH domain of the first Fab fragment or to the N-terminus of the VH domain of the domain exchanged Fab fragment, and
wherein the first antigen or the second antigen is human CD3.

In another likewise preferred embodiment the TCB comprises
a) a first and a second Fab fragment that each specifically bind to a first antigen,
b) one domain exchanged Fab fragment that specifically binds to a second antigen in which the VH and the VL domain are exchanged for each other,
c) one Fc-region comprising a first heavy chain Fc-region polypeptide and a second heavy chain Fc-region polypeptide,
wherein the C-terminus of CH1 domain of the first Fab fragment is connected to the N-terminus of one of the heavy chain Fc-region polypeptides and the C-terminus of the CH1-domain of the domain exchanged Fab fragment is connected to the N-terminus of the other heavy chain Fc-region polypeptide, and
wherein the C-terminus of the CH1 domain of the second Fab fragment is connected to the N-terminus of the VH domain of the first Fab fragment or to the N-terminus of the VL domain of the domain exchanged Fab fragment, and
wherein the first antigen or the second antigen is human CD3.

The term “valent” as used within the current application denotes the presence of a specified number of binding sites in a (antibody) molecule. As such, the terms “bivalent”, “tetravalent”, and “hexavalent” denote the presence of two binding site, four binding sites, and six binding sites, respectively, in a (antibody) molecule. The bispecific antibodies as reported herein as reported herein are in one preferred embodiment “bivalent”.

The term “variable region” or “variable domain” refer to the domain of an antibody heavy or light chain that is involved in binding of the antibody to its antigen. The variable domains of the heavy chain and light chain (VH and VL, respectively) of an antibody generally have similar structures, with each domain comprising four framework regions (FRs) and three hypervariable regions (HVRs) (see, e.g., Kindt, T. J. et al. Kuby Immunology, 6th ed., W.H. Freeman and Co., N.Y. (2007), page 91). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano, S. et al., J. Immunol. 150 (1993) 880-887; Clackson, T. et al., Nature 352 (1991) 624-628).

The terms “variant”, “modified antibody”, and “modified fusion polypeptide” denotes molecules that have an amino acid sequence that differs from the amino acid sequence of a parent molecule. Typically, such molecules have one or more alterations, insertions, or deletions. In certain embodiments, the modified antibody or the modified fusion polypeptide comprises an amino acid sequence comprising at least a portion of an Fc-region that is not naturally occurring. Such molecules have less than 100% sequence identity with the parent antibody or parent fusion polypeptide. In certain embodiments, the variant antibody or the variant fusion polypeptide has an amino acid sequence that has from about 75% to less than 100% amino acid sequence identity with the amino acid sequence of the parent antibody or parent fusion polypeptide, especially from about 80% to less than 100%, especially from about 85% to less than 100%, especially from about 90% to less than 100%, and especially from about 95% to less than 100%. In certain embodiments, the parent antibody or the parent fusion polypeptide and the variant antibody or the variant fusion polypeptide differ by one (a single), two or three amino acid residue(s).

A “primary human endothelial cell” is a human cell that has been directly isolated from its source, organ, tissue or blood, using enzymatic or mechanical methods. Primary cells are not immortalized. Once isolated, they are placed in an artificial environment, such as, e.g., in plastic or glass containers, in a specialized medium containing essential nutrients and growth factors to support proliferation. Primary cells can be of two types—adherent cells or cells growing in suspension. Adherent cells require attachment for growth and are said to be anchorage-dependent cells. The adherent cells are usually derived from tissues of organs. Suspension cells do not require attachment for growth and are said to be anchorage-independent cells. Most suspension cells are isolated from blood.

The term “pH-sensitive fluorescent dye” denotes a dye that has different fluorescence intensity or emission wavelengths at physiological pH of about pH 7.4 and at lysosomal pH of about pH 4.5.

### II. Antibodies In Vivo

Since IgG molecules are divalent, a single IgG molecule can neutralize up to two antigen molecules. For neutralizing antibodies, there are two types of target antigens: soluble-type antigens, which are present in plasma, and membrane-bound antigens, which are expressed on the surface of cells.

In case the antigen is a membrane-bound antigen, an administered therapeutic antibody binds to the membrane-bound antigen on the cellular surface. Subsequently the antibody is taken up into endosomes within the cell by internalization together with the membrane-bound antigen bound by the antibody. Thereafter, the antibody, which is still binding to the antigen moves to a lysosome where it is degraded together with the antigen. The elimination of an antibody from the plasma mediated by internalization by membrane-bound antigen is denoted as antigen-dependent elimination. This has been reported for different antibody molecules (see, e.g., Drug Discov. Today, 11 (2006) 81-88). Since a single IgG antibody molecule binds to two antigen molecules when it bivalently binds to antigens, and is then internalized and directly degraded by lysosome, a single ordinary IgG antibody cannot neutralize two or more antigen molecules.

The reason for the long retention (slow elimination) of IgG molecules in plasma is FcRn, known as an IgG molecule salvage receptor (see, e.g., Nat. Rev. Immunol. 7 (2007) 715-725). IgG molecules that have been taken up into endosomes by pinocytosis bind to FcRn expressed in endosomes under intraendosomal acidic conditions. IgG molecules bound to FcRn move to the cell surface where they dissociate from FcRn under neutral conditions of blood plasma. IgG molecules unable to bind to FcRn proceed into lysosomes where they are degraded.

If the IgG antibody, when taken up into intracellular endosomes by internalization, dissociates from the antigen under intraendosomal acidic conditions, the dissociated antibody can bind to FcRn also present in the endosome. Thereby, the IgG molecule dissociated from the antigen and bound by FcRn is transferred to the cell surface and released under pH neutral conditions in the plasma from FcRn. Thereby the antibody is recycled into the plasma. The IgG molecule that has returned to the plasma is able to bind to new antigens again. The repetition of this process allows a single IgG molecule to repeatedly bind to antigens, thereby enabling neutralization of a multiple antigens with a single IgG molecule.

In case of a soluble antigen, an administered therapeutic antibody binds to the antigen in the plasma, and remains in the plasma in the form of an antigen-antibody complex. As is the case with IgG molecules not bound to antigens, IgG molecules bound to antigens in the plasma are taken up into endosomes by pinocytosis. There they can bind to FcRn expressed in endosomes under intraendosomal acidic conditions. The IgG molecules bound to FcRn moves to the cell surface and then dissociate from the FcRn under neutral conditions in the plasma. If the IgG molecules can dissociate from the antigen under intraendosomal acidic conditions, the dissociated antigen will not be able to bind to FcRn and thereby may be degraded by lysosomes. Since the IgG molecules that have returned to the plasma have already dissociated from the antigen in endosomes, they are able to bind to a new antigen again in the plasma. The repetition of this process allows a single IgG molecule to repeatedly bind to soluble antigens. This enables a single IgG molecule to neutralize multiple antigens.

Thus, regardless of whether the antigen is a membrane-bound antigen or soluble antigen, if the dissociation of the IgG antibody from the antigen is possible under intraendosomal acidic conditions, a single IgG molecule can repeatedly neutralize antigens.

More specifically, a single IgG molecule that strongly binds to an antibody at the cell surface pH of 7.4 and weakly binds to the antigen at the intraendosomal pH of 5.5 to 6.0 may be able to neutralize a multiple antigens and thereby improve the pharmacokinetics (the intraendosomal pH has been reported to be typically pH 5.5 to 6.0 (see, e.g., Nat. Rev. Mol. Cell. Biol. 5 (2004) 121-132)).

In general, protein-protein interactions consist of hydrophobic interaction, electrostatic interaction and hydrogen bonding, and the binding strength is typically expressed as a binding constant (affinity) or apparent binding constant (avidity). pH-dependent binding, whose binding strength varies between neutral conditions (pH 7.4) and acidic conditions (pH 5.5 to 6.0), is present in naturally-occurring protein-protein interactions. For example, the above-mentioned binding between IgG molecules and FcRn known as a salvage receptor for IgG molecules is strong under acidic conditions (pH 5.5 to 6.0) but remarkably weak under neutral conditions (pH 7.4). It has been reported that the pH-dependent binding of the above-described IgG-FcRn interaction is associated with histidine residues present in IgG (see, e.g., Mol. Cell. 7 (2001) 867-877).

### III. Methods According to the Invention

Herein is reported a novel method for the estimation of the clearance of therapeutic antibodies in humans using a novel human primary cell-based in vitro assay. This Large molecule Unspecific Clearance Assay (LUCA) provides for an in vitro-based method to assess and predict PK properties for therapeutic antibodies.

The current invention is based, at least in part, on the finding that the sum of uptake and recycling of an antibody into primary human endothelial cells in vitro can be used as surrogate for estimating non-specific clearance of said antibody in vivo.

The current invention is based, at least in part, on the finding that only primary human endothelial cells can be used in predicting the in vivo clearance from an in vitro experiment as non-primary endothelial cells do not show the same correlation and, thus, are not suitable for this purpose. With said non-primary endothelial cells no differentiation between different antibodies can be achieved (compare FIGS. 1 and 2). A scheme of the method according to the current invention is depicted in FIG. 4.

Thus, the current invention comprises a method for determining or estimating the non-specific (non-target mediated) clearance (rate) of an antibody comprising the following steps:
a) incubating the antibody, which is conjugated to a pH-sensitive fluorescent dye, with primary human endothelial cells, and
b) determining the (intracellular) fluorescence intensity of the primary human endothelial cells of step a) (after a defined incubation time),
   whereby an increase of the (intracellular) fluorescence intensity of the primary human endothelial cells as determined in step b) above background level (i.e. the (intracellular) fluorescence of the primary human endothelial cells that had not been incubated with the antibody) is indicative for non-specific clearance of the antibody.

The current invention is based, at least in part, on the finding that the sum of uptake and recycling of an antibody into primary human endothelial cells in vitro can be used as surrogate for estimating non-specific clearance of said antibody in vivo.

The current invention is based, at least in part, on the finding that only primary human endothelial cells can be used for predicting the in vivo clearance from an in vitro experiment as non-primary endothelial cells do not show the same correlation and, thus, are not suitable for this purpose. With said non-primary endothelial cells no differentiation between different antibodies can be achieved (see FIGS. 1 and 2).

The current invention is based, at least in part, on the finding that the uptake of an antibody by pinocytosis and its routing to the lysosomal compartment without being recycled by FcRn of a primary endothelial cell accounts for the biggest contribution to the primary endothelial cell's fluorescence.

In FIGS. 1 and 2 a comparison of the time course of fluorescence intensity of different antibodies, which have been labelled with the same pH-sensitive fluorescent dye, during incubation with endothelial cells (human microvascular endothelial cells, HMEC1; FIG. 1) and during incubation with primary endothelial cells (human primary liver endothelial cells; FIG. 2) is shown. As can be seen from FIG. 1, for five of the seven antibodies no differentiation is possible when simple endothelial cells are used. In contrast thereto, for all seven antibodies a differentiation is possible when primary endothelial cells are use (see FIG. 2).

The labelled antibodies have been analyzed by heparin- and FcRn-chromatography. In the following Table exemplary retention times of the non-labelled and labelled antibodies are shown. It can be seen that the labelling does not alter the heparin- and FcRn-binding properties of the antibodies. For an antibody to be reliable within the assay according to the current invention, the difference from the geo-mean was expected to be below 15%.

TABLE 1
Retention times of the non-labelled and labelled antibodies
on human heparin and human FcRn chromatography columns.

| | Heparin | Heparin | FcRn | FcRn |
|---|---|---|---|---|
| | (rel. to | (rel. to | (rel. to | (rel. to |
| antibody: | pTau- | pTau- | Her3- | Her3- |
| name or specificity | labeled) | unlabeled) | labeled) | unlabeled) |
| Adalimumab | 0.79 | 0.81 | 0.84 | 0.93 |
| anti-human alpha | 1.19 | 1.2 | 1.85 | 1.84 |
| synuclein antibody | | | | |
| Avelumab | 0.6 | 0.61 | 1.25 | 1.27 |
| TCB-1 | 0.79 | 0.79 | 3.52 | 3.55 |
| TCB-2 | 1 | 1.01 | 1.46 | 1.56 |
| anti-Her3 antibody | 0.65 | 0.63 | 1.06 | 1.01 |
| Ixekizumab | 0.85 | 0.86 | 1.33 | 1.42 |
| Motavizumab WT | 0.69 | 0.71 | 0.42 | 0.50 |
| Motavizumab-YTE | 0.7 | 0.69 | 4.88 | 4.52 |
| Nivolumab | 0.62 | 0.63 | 0.39 | 0.49 |
| Ofatumumab | 0.57 | 0.58 | 0.67 | 0.72 |
| Palivizumab | 0.64 | 0.65 | 0.28 | 0.31 |
| anti-human p-selectin | 0.6 | 0.61 | 0.01 | 0.12 |
| antibody (IgG4) | | | | |
| anti-human phospho | 1.00 | 1.00 | 1.18 | 1.13 |
| tau 422 antibody | | | | |
| Reslizumab | 0.22 | 0.24 | 0.41 | 0.50 |
| Ustekinumab | 0.67 | 0.68 | 0.41 | 0.48 |
| Vedolizumab | 0.52 | 0.54 | 0.29 | 0.33 |

The fluorescence label used in the method according to the invention can be any pH-dependent fluorescent dye that has a shift of fluorescence intensity between physiological pH of about 7 to acidic pH in the range of pH 4 to 5 of about 10-fold, preferably about 25-fold, and most preferably of about 50-fold.

Exemplary suitable dyes are the pHAb Dyes marketed by Promega. These dyes are pH sensor dyes that have very low fluorescence at pH>7 and a dramatic increase in fluorescence as the pH of the solution becomes acidic. pHAb Dyes have excitation maxima (Ex) at 532 nm and emission maxima (Em) at 560 nm. pHAb Dyes are available in two reactive forms suitable for antibody conjugation: pHAb Amine Reactive Dye and pHAb Thiol Reactive Dye. pHAb Amine Reactive Dye has a succinimidyl ester group that reacts with primary amines available on the lysine amino acids on the antibody. pHAb Thiol Reactive Dye has a maleimido group that reacts with thiols. This maleimido group is expected to be conjugated to the antibody after the cysteine disulfide bonds in the hinge region of the antibody are reduced to thiols through the use of a reducing agent, such as DTT or TCEP. pHAb Dyes retain their fluorescent response to decreases in pH after conjugation to antibody.

A not-suitable dye is Invitrogen's Click-iT™ pHrodo™ iFL Red sDIBO Alkyne. This dye shows only a small change in fluorescence intensity upon pH changes of only 2 to 3-fold.

One suitable linker is sulfo DBCO-PEG4-Amine marketed by ClickChemistryTools. Sulfo DBCO-PEG4-Amine is a water-soluble reagent used to derivatize carboxyl-containing molecules or activated esters (e.g. NHS esters) with DBCO moiety through a stable amide bond. The hydrophilic sulfonated spacer arm improves water solubility of DBCO derivatized molecules, making it in many cases completely soluble in aqueous media. The PEG spacer arm provides a long and flexible connection. The conjugation is achieved by azide-activation of the antibody and reaction with the DBCO moiety using a Click-chemistry-reaction.

In the following, the invention is exemplified using a pHAb dye and a conjugation using a sulfo DBCO-PEG4-Amine linker. Any other dye exhibiting the above outlined characteristics or linker or conjugation chemistry not interfering with the binding properties of the antibody as well as the pH-dependent fluorescence properties of the dye can likewise be used. This is simply presented as exemplification of the invention and shall not be construed as a limitation. The true scope is set forth in the appended claims.

The structure of this exemplary labelled antibody is shown in FIG. 3.

The mean fluorescent intensity (MFI, more specifically the geometric mean fluorescence intensity) of the internalized antibodies was acquired using FACS with excitation at 488 nm and detection at 585/540 nm. The exact same conditions, gains and gates were used for all times points (i.e. 2 and 4 hours). Data extraction was performed using the FloJo_V10 software. Values of the negative control was subtracted from all geometric mean values followed by normalization to the dye antibody ratio (DAR). The normalized geometric-mean values from each antibody were plotted as linear regression curve using GraphPad Prism to extract the slope (Geo Mean MFI/min for 120 and 240 min and comprising the point of origin, i.e. 0/0). Two antibodies were used to normalize the slopes: Motavizumab with the mutation M252Y/S254T/T256E was set to 0 and a TCB was set to 1. These antibodies were selected as they span a sufficient range of rates. The final slopes were plotted against the respective in vivo human, cynomolgus and hFcRn Tg32+/+ mouse clearance values using the TIBCO Spotfire software. The respective plots for human, cynomolgus and human FcRn transgenic mouse with different antibodies including those of Table 1 are shown in FIGS. 5 to 7.

FIG. 8 shows that the method according to the current invention can also be used to determine in vivo clearance of Fc-region variants of IgGs. This further indicates that FcRn recycling is appropriately captured in the method according to the current invention.

FIG. 9 shows the dependency of the fluorescence from the incubation time. It can be seen that the linear range is at least up to 24 hours.

In certain embodiments, the method according to the current invention is a method for estimating or determining the in vivo clearance rate of an antibody in human or cynomolgus or mouse comprising the following steps:
a) incubating separately the antibody and at least a first and a second reference antibody, which are conjugated to the same pH-sensitive fluorescent dye, with primary human endothelial cells for at least two and four hours, and determining the geometric mean intracellular fluorescence intensity of the primary human endothelial cells for each incubation time, optionally the cell are washed prior to the determination of the intracellular fluorescence intensity to removed adherent fluorescently labelled antibody,
b) determining the geometric mean intracellular fluorescence intensity of the primary human endothelial cells incubated without any labelled antibody at the same time points as in a), optionally the cell are washed prior to the determination of the intracellular fluorescence intensity to removed adherent fluorescent compounds,
c) determining the relative normalized intracellular fluorescence intensity rate by
   i) subtracting from each of the geometric mean intracellular fluorescence intensities determined in a) for the antibody in question and the reference antibodies the geometric mean intracellular fluorescence intensity of the primary human endothelial cells determined at the same time point to obtain corrected geometric mean intracellular fluorescence intensities,
   ii) dividing separately the corrected geometric mean intracellular fluorescence intensities obtained in 2) of the antibody in question and the reference antibodies by the number of fluorescent dye molecules present in the respective antibody to obtain a normalized geometric mean intracellular fluorescence intensity,
   iii) determining the slope of the best fit straight line (i.e. a linear regression curve y=s*x+b with y=normalized geometric mean (intracellular) fluorescence intensity, s=slope, x=time and b=y-axis intersection) for each of the antibody in question and the reference antibodies based on the group of values consisting of aa) the normalized geometric mean intracellular fluorescence intensities for each incubation time of a) as determined in ii) and bb) the point of origin,
   iv) normalizing the slope of the best-fit straight line of the antibody in question as follows:

$$normalized slope ( antibody in question ) = \frac{s l ope ( antibody in question ) - s l ope ( reference antibody with slower clearance )}{slope ( reference antibody with faster clearance ) - slope ( reference antibody with slower clearance )}$$

whereby the in vivo clearance rate of the antibody in human or cynomolgus or mouse is the clearance rate of the first reference antibody in human or cynomolgus or mouse multiplied by the relative normalized intracellular fluorescence intensity rate.

It has been found that by using a relative normalized rate (intracellular) fluorescence intensity (rate) intraassay and intraday deviations can be minimized.

By using the alignment according to the invention between relative normalized intracellular fluorescence intensity rates and in vivo-determined clearance rates an in vitro-in vivo-correlation has been established. This correlation is not dependent on the specific antibodies used during the generation thereof. Likewise, other antibodies with known in vivo clearance rates can be used.

By using the relative normalized intracellular fluorescence intensity rate determined for an antibody with unknown in vivo clearance rate as x-value in the in vivo-in vitro-correlation according to the invention the in vivo clearance of the antibody with not determined in vivo clearance rate can be estimated as y-value.

The following examples, sequences and figures are provided to aid the understanding of the present invention, the true scope of which is set forth in the appended claims.

It is understood that modifications can be made in the procedures set forth without departing from the spirit of the invention.

### DESCRIPTION OF THE FIGURES

FIG. 1 Time course of fluorescence intensity of different antibodies, which have been labelled with the same pH-sensitive fluorescent dye, during incubation with human microvascular endothelial cells; 1=anti-human phospho Tau 422 antibody; 2=anti-CD44 antibody; 3=Olaratumab; 4=anti-CD20 antibody (1); 5=Avelumab; 6=anti-human alpha-synuclein antibody; 7=anti-CD20 antibody (2).

FIG. 2 Time course of fluorescence intensity of different antibodies, which have been labelled with the same pH-sensitive fluorescent dye, during incubation with human primary liver endothelial cells; 1=anti-human phospho Tau 422 antibody; 2=anti-CD44 antibody; 3=Olaratumab; 4=anti-CD20 antibody (1); 5=Avelumab; 6=anti-human alpha-synuclein antibody; 7=anti-CD20 antibody (2).

FIG. 3 Scheme of a fluorescently labelled antibody used in the method according to the current invention; pHAb dye conjugated via a sulfo DBCO-PEG4-Amine linker to the antibody.

FIG. 4 Scheme of the method according to the current invention.

FIG. 5 Corrected mean fluorescent intensity (MFI, more specifically the geometric mean) of the internalized antibodies as acquired using FACS were obtained by subtracting the negative control followed by normalization (division) with the dye antibody ratio (DAR). The corrected and normalized geo-mean values from each antibody were plotted as linear regression curve and the slope (Geo Mean MFI/min for 120 and 240 min) was extracted. Two standard antibodies were selected to normalize the slopes: Motavizumab-YTE was set to 0 and a TCB was set to 1. The final slopes were plotted against in vivo human clearance values. If different clearance values were available, dose linear clearance describing the unspecific clearance of the molecule, was used.

FIG. 6 Corrected mean fluorescent intensity (MFI, more specifically the geometric mean) of the internalized antibodies as acquired using FACS were obtained by subtracting the negative control followed by normalization (division) with the dye antibody ratio (DAR). The corrected and normalized geo-mean values from each antibody were plotted as linear regression curve and the slope (Geo Mean MFI/min for 120 and 240 min) was extracted. Two standard antibodies were selected to normalize the slopes: Motavizumab-YTE was set to 0 and a TCB was set to 1. The final slopes were plotted against in vivo cynomolgus clearance values. If different clearance values were available, dose linear clearance describing the unspecific clearance of the molecule, was used.

FIG. 7 Corrected mean fluorescent intensity (MFI, more specifically the geometric mean) of the internalized antibodies as acquired using FACS were obtained by subtracting the negative control followed by normalization (division) with the dye antibody ratio (DAR). The corrected and normalized geo-mean values from each antibody were plotted as linear regression curve and the slope (Geo Mean MFI/min for 120 and 240 min) was extracted. Two standard antibodies were selected to normalize the slopes: Motavizumab-YTE was set to 0 and a TCB was set to 1. The final slopes were plotted against in vivo hFcRn Tg32+/+ mouse clearance values.

FIG. 8 Fc variants of IgGs show same in vitro-in vivo correlation as wt Fc IgGs.

FIG. 9 Time course of mean fluorescence intensity of primary human endothelial cells incubated with a monospecific bivalent antibody.

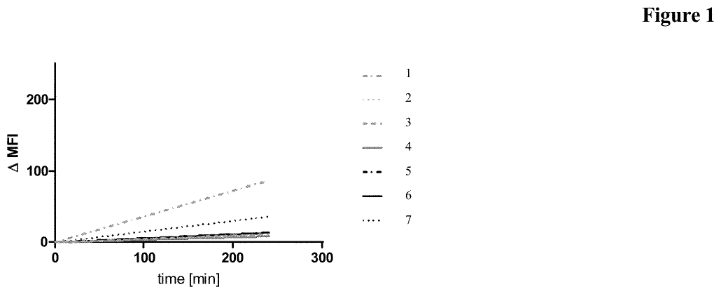

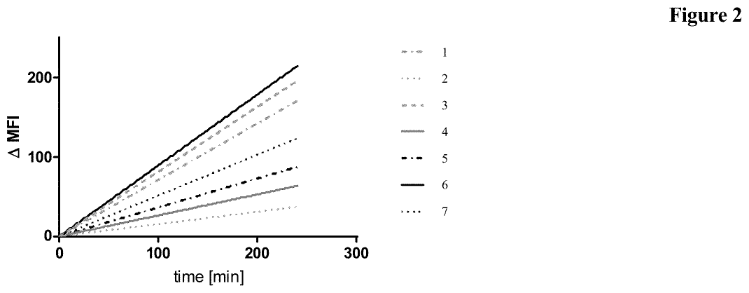

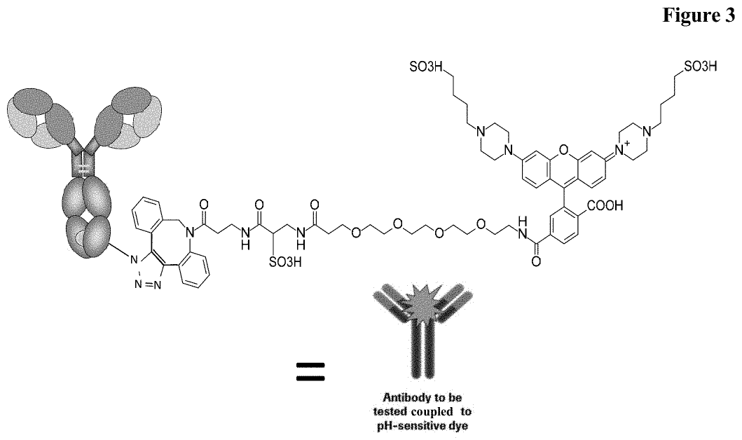

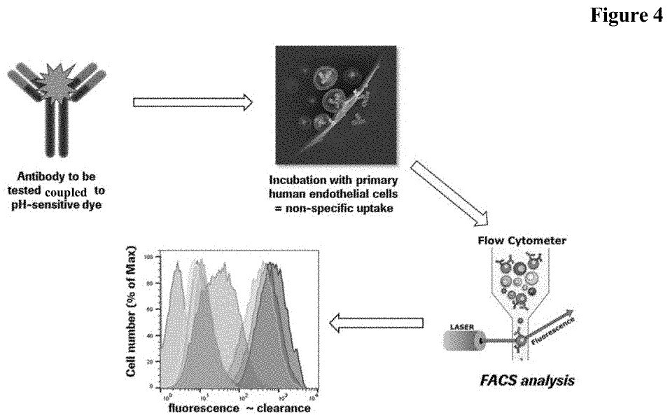

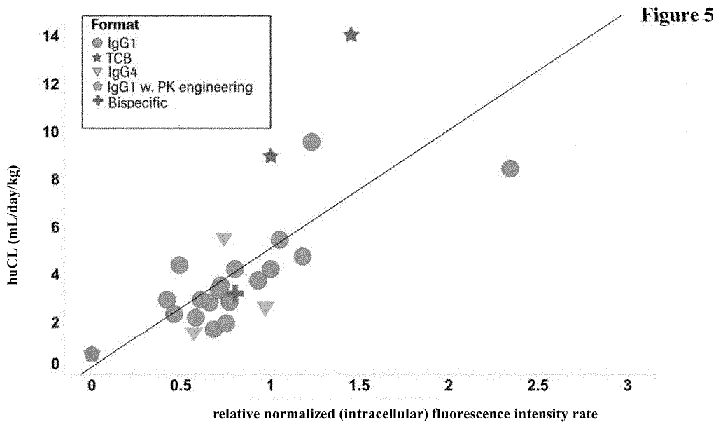

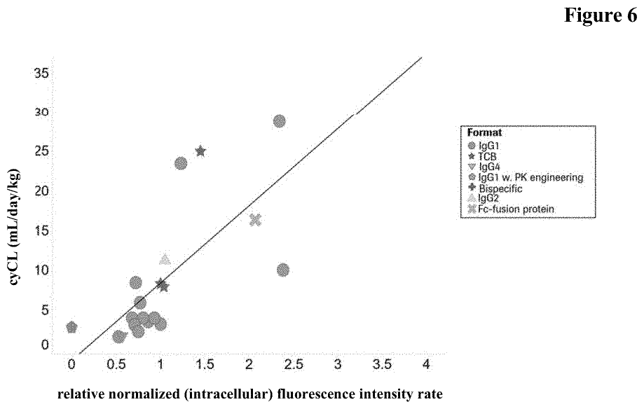

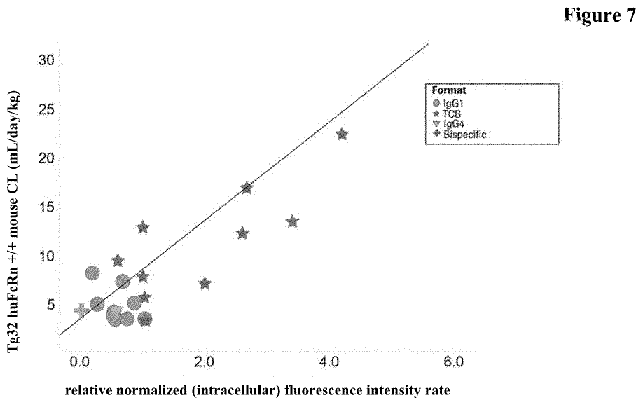

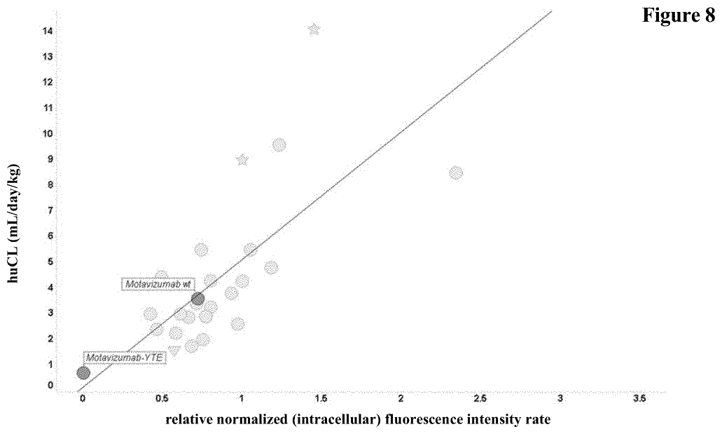

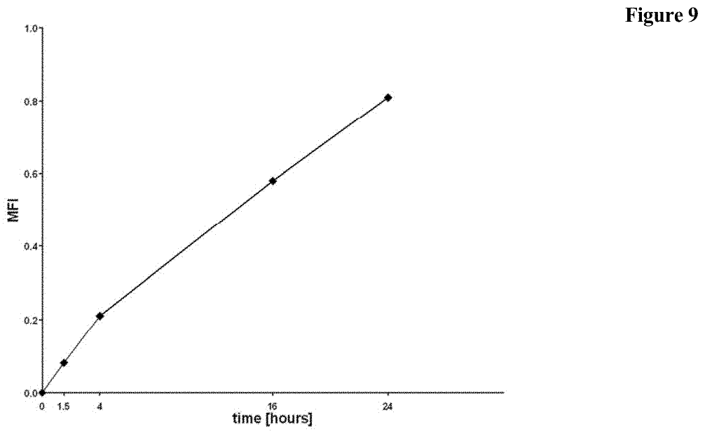

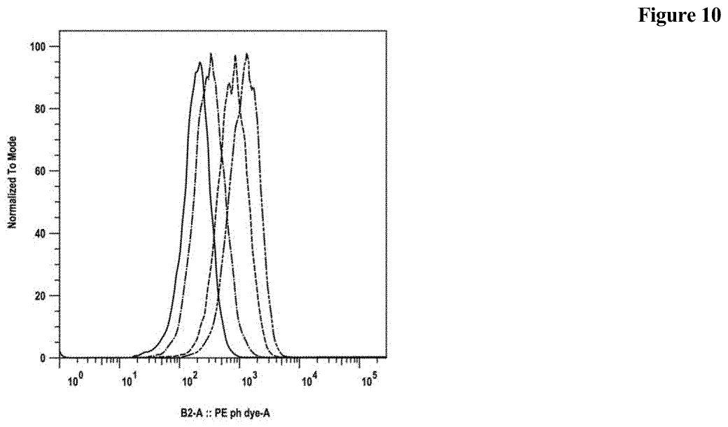

FIG. 10 Flow cytometry analysis of primary human liver-derived endothelial cells. Endothelial cells were incubated with antibodies, prior labeled with a pHAb amine reactive dye (532 nm): the low clearing antibody Motavizumab-YTE (solid line), two medium clearing bispecific antibodies (dash-point-dash line and dashed line, respectively), as well as a high clearing bispecific antibody (dash-point-point-dash line). After 4 hours, the fluorescence intensity was recorded and cells were gated for singlet population, morphology and viability;
y-axis scaling is relative to the number of events,
x-axis scaling displays the intensity in the PE channel.

### EXAMPLES

### I. Materials and Methods

### Antibodies

The reference antibodies used in the experiments were an anti-pTau antibody that has the heavy chain amino acid sequence of SEQ ID NO: 01 and the light chain amino acid sequence of SEQ ID NO: 02 and an anti-Her 3 antibody that has the heavy chain amino acid sequence of SEQ ID NO: 03 and the light chain amino acid sequence of SEQ ID NO: 04.

Synthetic genes were produced at Geneart (Life technologies GmbH, Carlsbad, Calif., USA).

The monoclonal antibodies used herein were transiently expressed in HEK293 cells (see below) and purification was performed by protein A chromatography using standard procedures (see below).

The biochemical characterization included size exclusion chromatography (Waters BioSuite™ 250 7.8×300 mm, eluent: 200 mM KH₂PO₄, 250 mM KCl, pH 7.0) and analysis of the molecular weight distribution using the BioAnalyzer 2100 (Agilent technologies, Santa Clara, Calif., USA).

### Expression Plasmids

For the expression of the above described antibodies, variants of expression plasmids for transient expression (e.g. in HEK293-F) cells based either on a cDNA organization with or without a CMV-Intron A promoter or on a genomic organization with a CMV promoter were applied.

Beside the antibody expression cassette the plasmids contained:
an origin of replication which allows replication of this plasmid in E. coli,
a β-lactamase gene which confers ampicillin resistance in E. coli., and
the dihydrofolate reductase gene from Mus musculus as a selectable marker in eukaryotic cells.

The transcription unit of the antibody gene was composed of the following elements:
unique restriction site(s) at the 5′ end
the immediate early enhancer and promoter from the human cytomegalovirus,
followed by the Intron A sequence in the case of the cDNA organization,
a 5′-untranslated region of a human antibody gene,
an immunoglobulin heavy chain signal sequence,
the human antibody chain either as cDNA or as genomic organization with the immunoglobulin exon-intron organization
a 3′ non-translated region with a polyadenylation signal sequence, and
unique restriction site(s) at the 3′ end.

The fusion genes comprising the antibody chains were generated by PCR and/or gene synthesis and assembled by known recombinant methods and techniques by connection of the according nucleic acid segments e.g. using unique restriction sites in the respective plasmids. The subcloned nucleic acid sequences were verified by DNA sequencing. For transient transfections larger quantities of the plasmids were prepared by plasmid preparation from transformed E. coli cultures (Nucleobond AX, Macherey-Nagel).

### Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

### Transient Transfections in HEK293-F System

The antibodies were generated by transient transfection with the respective plasmids (e.g. encoding the heavy chain, as well as the corresponding light chain) using the HEK293-F system (Invitrogen) according to the manufacturer's instruction. Briefly, HEK293-F cells (Invitrogen) growing in suspension either in a shake flask or in a stirred fermenter in serum-free FreeStyle™ 293 expression medium (Invitrogen) were transfected with a mix of the respective expression plasmids and 293Fectin™ or fectin (Invitrogen). For 2 L shake flask (Corning) HEK293-F cells were seeded at a density of 1*10⁶ cells/mL in 600 mL and incubated at 120 rpm, 8% CO₂. The day after the cells were transfected at a cell density of ca. 1.5*10⁶ cells/mL with ca. 42 mL mix of A) 20 mL Opti-MEM (Invitrogen) with 600 μg total plasmid DNA (1 μg/mL) encoding the heavy chain, respectively and the corresponding light chain in an equimolar ratio and B) 20 ml Opti-MEM+1.2 mL 293 fectin or fectin (2 μL/mL). According to the glucose consumption glucose solution was added during the course of the fermentation. The supernatant containing the secreted antibody was harvested after 5-10 days and antibodies were either directly purified from the supernatant or the supernatant was frozen and stored. Some of the antibodies that have been produced accordingly:

| antibody | format |
|---|---|
| Adalimumab | IgG1 |
| anti-human alpha synuclein antibody | IgG1 |
| Avelumab | IgG1 |
| TCB-1 (reference TCB) | TCB |
| CD-Ag-1/CD-AG-2 bispecific antibody | CrossMab |
| TCB-2 | TCB |
| wild-type IgG with sortase tag | IgG1 |
| non-binding antibody with sortase tag | IgG1 |
| TCB-3 | TCB |
| TCB-4 | TCB |
| anti-Her3 antibody | IgG1 |
| Ixekizumab | IgG4 |
| TCB-5 | TCB |
| TCB-6 | TCB |
| Motavizumab wt | IgG1 |
| Motavizumab-YTE | IgG1 |
| Nivolumab | IgG1 |
| Ofatumumab | IgG1 |
| Palivizumab | IgG1 |
| anti-human p(422)Tau antibody | IgG1 |
| Reslizumab | IgG4 |
| Ustekinumab | IgG1 |
| Vedolizumab | IgG1 |

### Purification

Antibodies were purified from cell culture supernatants by affinity chromatography using MabSelectSure-Sepharose™ (GE Healthcare, Sweden), hydrophobic interaction chromatography using butyl-Sepharose (GE Healthcare, Sweden) and

Superdex 200 size exclusion (GE Healthcare, Sweden) chromatography.

Briefly, sterile filtered cell culture supernatants were captured on a MabSelectSuRe resin equilibrated with PBS buffer (10 mMNa₂HPO₄, 1 mM KH₂PO₄, 137 mM NaCl and 2.7 mM KCl, pH 7.4), washed with equilibration buffer and eluted with 25 mM sodium citrate at pH 3.0. The eluted antibody fractions were pooled and neutralized with 2 M Tris, pH 9.0. The antibody pools were prepared for hydrophobic interaction chromatography by adding 1.6 M ammonium sulfate solution to a final concentration of 0.8 M ammonium sulfate and the pH adjusted to pH 5.0 using acetic acid. After equilibration of the butyl-Sepharose resin with 35 mM sodium acetate, 0.8 M ammonium sulfate, pH 5.0, the antibodies were applied to the resin, washed with equilibration buffer and eluted with a linear gradient to 35 mM sodium acetate pH 5.0. The antibody containing fractions were pooled and further purified by size exclusion chromatography using a Superdex 200 26/60 GL (GE Healthcare, Sweden) column equilibrated with 20 mM histidine, 140 mM NaCl, pH 6.0. The antibody containing fractions were pooled, concentrated to the required concentration using Vivaspin ultrafiltration devices (Sartorius Stedim Biotech S.A., France) and stored at −80° C.

Purity and antibody integrity were analyzed after each purification step by CE-SDS using microfluidic Labchip technology (Caliper Life Science, USA). Five μl of protein solution was prepared for CE-SDS analysis using the HT Protein Express Reagent Kit according manufacturer's instructions and analyzed on LabChip GXII system using a HT Protein Express Chip. Data were analyzed using LabChip GX Software.

### Mice

B6.Cg-Fcgrt^{tm/Dcr} Tg(FCGRT)276Dcr mice deficient in mouse FcRn α-chain gene, but hemizygous transgenic for a human FcRn α-chain gene (muFcRn−/− huFcRn tg +/−, line 276) were used for the pharmacokinetic studies. Mouse husbandry was carried out under specific pathogen free conditions. Mice were obtained from the Jackson Laboratory (Bar Harbor, Me., USA) (female, age 4-10 weeks, weight 17-22 g at time of dosing). All animal experiments were approved by the Government of Upper Bavaria, Germany (permit number 55.2-1-54-2532.2-28-10) and performed in an AAALAC accredited animal facility according to the European Union Normative for Care and Use of Experimental Animals. The animals were housed in standard cages and had free access to food and water during the whole study period.

### Pharmacokinetic Studies

A single dose of antibody was injected i.v. via the lateral tail vein at a dose level of 5 mg/kg. The mice were divided into 3 groups of 6 mice each to cover 9 serum collection time points in total (at 0.08, 2, 8, 24, 48, 168, 336, 504 and 672 hours post dose). Each mouse was subjected twice to retro-orbital bleeding, performed under light anesthesia with Isoflurane™ (CP-Pharma GmbH, Burgdorf, Germany); a third blood sample was collected at the time of euthanasia. Blood was collected into serum tubes (Microvette 500Z-Gel, Sarstedt, Nümbrecht, Germany). After 2 h incubation, samples were centrifuged for 3 min at 9.300 g to obtain serum. After centrifugation, serum samples were stored frozen at −20° C. until analysis.

### Determination of Human Antibody Serum Concentrations

Concentrations of antibodies in murine serum were determined by specific enzyme-linked immunoassays. Biotinylated capture reagent specific for each of the antibodies and digoxygenin-labeled anti-human-Fc mouse monoclonal antibody (Roche Diagnostics, Penzberg, Germany) were used for capturing and detection, respectively. Streptavidin-coated microtiter plates (Roche Diagnostics, Penzberg, Germany) were coated with biotinylated capture reagent diluted in assay buffer (Roche Diagnostics, Penzberg, Germany) for 1 h. After washing, serum samples were added at various dilutions followed by another incubation step for 1 h. After repeated washings, bound antibodies were detected by subsequent incubation with detection antibody, followed by an anti-digoxygenin antibody conjugated to horseradish peroxidase (HRP; Roche Diagnostics, Penzberg, Germany). ABTS (2,2′Azino-di[3-ethylbenzthiazoline sulfonate]; Roche Diagnostics, Germany) was used as HRP substrate to form a colored reaction product. Absorbance of the resulting reaction product was read at 405 nm with a reference wavelength at 490 nm using a Tecan sunrise plate reader (Mannedorf, Switzerland).

All serum samples, positive and negative control samples were analyzed in duplicates and calibrated against reference standard.

### PK Analysis

The pharmacokinetic parameters were calculated by non-compartmental analysis using WinNonlin™ 1.1.1 (Pharsight, CA, USA).

Briefly, area under the curve (AUC_{0-inf}) values were calculated by logarithmic trapezoidal method due to non-linear decrease of the antibodies and extrapolated to infinity using the apparent terminal rate constant λz, with extrapolation from the observed concentration at the last time point.

Plasma clearance was calculated as Dose rate (D) divided by AUC_{0-inf}. The apparent terminal half-life (T1/2) was derived from the equation T1/2=ln2/λz.

### Example 1

### Cynomolgus SDPK Studies

The pharmacokinetics of the test compounds was determined in cynomolgus monkeys following single intravenous administration at dose levels ranging from 0.3 mg/kg to 150 mg/kg. Serial blood samples were collected from the monkeys over several weeks and serum/plasma was prepared from the collected blood samples. Serum/plasma levels of test compounds were determined by ELISA. In case of linear pharmacokinetics pharmacokinetic parameter were determined by standard non-compartmental methods. Clearance was calculated according to following formula:

Clearance=Dose/Area under concentration-time curve

In cases of non-linear pharmacokinetics the linear fraction of the clearance was determined via following alternative methods: Either clearance values were estimated following IV administration at high dose levels, at which additional non-linear clearance pathways are virtually saturated. Alternatively, PK models comprising a linear and a non-linear, saturable clearance term were established. In these cases, the model-determined linear clearance fraction was used for correlations.

### Example 2

### Preparation of FcRn Affinity Column

### Expression of FcRn in HEK293 Cells

FcRn was transiently expressed by transfection of HEK293 cells with two plasmids containing the coding sequence of FcRn and of beta-2-microglobulin. The transfected cells were cultured in shaker flasks at 36.5° C., 120 rpm (shaker amplitude 5 cm), 80% humidity and 7% CO₂. The cells were diluted every 2-3 days to a density of 3 to 4*10⁵ cells/ml.

For transient expression, a 14 l stainless steel bioreactor was started with a culture volume of 81 at 36.5° C., pH 7.0±0.2, pO₂ 35% (gassing with N₂ and air, total gas flow 200 ml min⁻¹) and a stirrer speed of 100-400 rpm. When the cell density reached 20*10⁵ cells/ml, 10 mg plasmid DNA (equimolar amounts of both plasmids) was diluted in 400 ml Opti-MEM (Invitrogen). 20 ml of 293fectin (Invitrogen) was added to this mixture, which was then incubated for 15 minutes at room temperature and subsequently transferred into the fermenter. From the next day on, the cells were supplied with nutrients in continuous mode: a feed solution was added at a rate of 500 ml per day and glucose as needed to keep the level above 2 g/l. The supernatant was harvested 7 days after transfection using a swing head centrifuge with 11 buckets at 4000 rpm for 90 minutes. The supernatant (13 L) was cleared by a Sartobran P filter (0.45 μm+0.2 μm, Sartorius) and the FcRn beta-2-microglobulin complex was purified therefrom.

### Biotinylation of Neonatal Fc Receptor

3 mg FcRn beta-2-microglobulin complex were solved/diluted in 5.3 mL 20 mM sodium dihydrogenphosphate buffer containing 150 mM sodium chloride and added to 250 μL PBS and 1 tablet complete protease inhibitor (complete ULTRA Tablets, Roche Diagnostics GmbH). FcRn was biotinylated using the biotinylation kit from Avidity according to the manufacturer instructions (Bulk BIRA, Avidity LLC). The biotinylation reaction was done at room temperature overnight.

The biotinylated FcRn was dialyzed against 20 mM MES buffer comprising 140 mM NaCl, pH 5.5 (buffer A) at 4° C. overnight to remove excess of biotin.

### Coupling to Streptavidin Sepharose

For coupling to streptavidin Sepharose, 1 mL streptavidin Sepharose (GE Healthcare, United Kingdom) was added to the biotinylated and dialyzed FcRn beta-2-microglobulin complex and incubated at 4° C. overnight. The FcRn beta-2-microglobulin complex derivatized Sepharose was filled a 4.6 mm×50 mm chromatographic column (Repligen). The column was stored in 80% buffer A and 20% buffer B (20 mM Tris(hydroxymethyl)aminomethane pH 8.8, 140 mM NaCl).

### Example 3

### Chromatography Using FcRn Affinity Column and pH Gradient

### Conditions:

column dimensions: 50 mm×4.6 mm
loading: 30 μg sample
buffer A: 20 mM MES, with 140 mM NaCl, adjusted to pH to pH 5.5
buffer B: 20 mM Tris/HCl, with 140 mM NaCl, adjusted to pH 8.8

30 μg of samples were applied onto the FcRn affinity column equilibrated with buffer A. After a washing step of 10 minutes in 20% buffer B at a flow rate of 0.5 mL/min, elution was performed with a linear gradient from 20% to 70% buffer B over 70 minutes. The UV light absorption at a wavelength of 280 nm was used for detection. The column was regenerated for 10 minutes using 20% buffer B after each run.

For the calculation of relative retention times, a standard sample (anti-Her3 antibody (SEQ ID NO: 03 and 04), oxidized for 18 hours with 0.02% hydrogen peroxide according to Bertoletti-Ciarlet, A., et al. (Mol. Immunol. 46 (2009) 1878-1882) was run at the beginning of a sequence and after each 10 sample injections.

Briefly, the antibody (at 9 mg/mL) in 10 mM sodium phosphate pH 7.0 was mixed with H₂O₂ to a final concentration of 0.02% and incubated at room temperature for 18 hours. To quench the reaction the samples were thoroughly dialyzed into pre-cooled 10 mM sodium acetate buffer pH 5.0.

### Example 4

### Chromatography Using Heparin Affinity Column and pH Gradient

### Conditions:

column dimensions: 50 mm×5.0 mm
loading: 20-50 μg sample
buffer A: 50 mM TRIS pH 7.4
buffer B: 50 mM TRIS pH 7.4, 1000 mM NaCl

20 to 50 μg of protein samples in low-salt buffer (<25 mM ionic strength) were applied to a TSKgel Heparin-5PW Glass column, 5.0×50 mm (Tosoh Bioscience, Tokyo/Japan), which was pre-equilibrated with buffer A at room temperature. Elution was performed with a linear gradient from 0-100% buffer B over 32 minutes at a flow rate of 0.8 mg/mL. The UV light absorption at a wavelength of 280 nm was used for detection.

### Example 5

### Examination of Antibody Internalization

The method is based on a previously reported method that detects the internalized antibody using homogeneous fluorescence imaging of a pH-activated probe, which enables maximum fluorescence signals of antibody under intracellular acidic conditions without any fluorescence signals detected in the extracellular environments (Li, Z., et al., Int. Immunopharm. 62 (2018) 299-308).

Briefly, the respective antibody was conjugated with pHAb Amine Reactive dye and then diluted with cell culture medium. Meanwhile, cells were seeded into a 6-well plate (1×10⁵ cells per well), and 100 μL of medium containing pHAb Amine Reactive dyes-conjugated antibody (final concentration of 10 μg/mL) was added into each well. After incubation at 37° C., the internalization of the antibody was measured by flow cytometry at different time points (0 h, 1.5 h, 2 h, 4 h, 5.5 h, and/or 24 h).

### Example 6

### Antibody Labeling

Antibodies were labeled using the SiteClick™ Antibody Azido Modification Kit (Thermo Fisher Scientific) according to the manufactures instructions. Briefly, N-linked galactose residues of the Fc-region were removed by β-galactosidase and replaced by an azide-containing galactose (GalNaz) via β-1,4-galactosyltransferase (GalT). This azide modification enables a copper-free conjugation of sDIBO-modified dyes. The pH-sensitive amine-reactive dye (523 nm) was purchased from Promega and coupled to a sulfo DBCO PEG4 amine. Antibodies were labeled with a molar dye excess of 2. Excess dye was removed using the Amicon® Ultra-2 Centrifugal Filter with a MWCO of 50 kDa (EMD Millipore, #UFC200324) and antibodies were re-buffered in 20 mM histidine buffer (pH 5.5). The concentration of the labeled antibodies [1] as well as the dye to antibody ratio (DAR) [2] was determined with a Nanodrop spectrometer at 280 nm (A₂₈₀ₙₘ) and 532 nm (A532 nm).

CAB=[A₂₈₀ₙₘ−[A₂₈₀ₙₘ*CFDye]]/ε_{mAb} [1]

DAR=[A₅₃₂ₙₘ*MW_{mAb}]/[c_{mAb}*ε_{Dye}] [2]

εDye=47225

CF_{Dye}=0.36

### Example 7

### Cell Maintenance and Preparation

Cryopreserved human liver-derived endothelial Cells (HLEC-P2) were purchased from Lonza (Lonza, #HLECP2). Cell were maintained in EBMT™-2 Endothelial Cell Growth Basal Medium-2 (Lonza, #CC-3156) supplemented with EGM™-2 MV Microvascular Endothelial Cell Growth Medium SingleQuots™ (Lonza, #CC-4176). Five days prior antibody treatment, cells were plated onto collagen I coated 100 mm culture dishes (Corning® BioCoat™, #354450) and two days prior treatment sub-cultured into collagen I coated 96-well plates (Corning® BioCoat™ #354407) at a cell density of 4×10⁴ cells/well to allow adherence for 48 hours. Medium was changed after 24 hours and cells were kept at 37° C. and 5% CO₂.

On the day of the experiment, cells were washed twice with 200 μl pre-warmed medium and subsequently incubated with 400 nM labeled antibody or 20 mM histidine buffer (pH 5.5) as negative control in medium. After 2 and 4 hours, the antibody solution was removed and cells were washed once with 200 μl ice-cold DPBS (without Mg and Ca) and detached by applying 100 μl Trypsin (with EDTA) for 2.5 minutes at 37° C. Trypsin was inactivated by the addition of 100 μl FACS Buffer (20% FCS, 1 mM EDTA in DPBS).

### Example 8

### Flow Cytometry and Pharmacokinetic Analysis

The mean fluorescent intensity (MFI, more specifically the geometric mean (geo-mean)) of the internalized antibodies was acquired using the MACSQuant® Analyzer 10 (Miltenyi Biotec) equipped with a laser to excite at 488 nm and a filter to collect emitted light at 585 nm/540 nm. The exact same conditions, gains and gates were used for both times points (2 hours and 4 hours). Data extraction was performed using the FloJo_V10 software. Values of the negative control was subtracted from all geo-mean values followed by normalization to the DAR. The normalized geo-mean values from each antibody were plotted as linear regression curve using GraphPad Prism to extract the slope (Geo Mean MFI/min for 120 and 240 min). Two standard antibodies were selected to normalize the slopes: Motavizumab-YTE was set to 0 and a TCB was set to 1. The final slopes were plotted against published in vivo human, cynomolgus and hFcRn Tg32+/+ mouse clearance values using the TIBCO Spotfire software.

### Example 9

### Quality Control

Biophysical binding properties are key determinants affecting clearance mechanisms. Therefore, it is important to assess, whether the binding affinities of the antibodies changed during the labeling process. Heparin chromatography and neonatal Fc-receptor binding has been previously shown to allow prediction of antibody clearance in vitro (Kraft, T. E., et. al., MABS 12 (2020) e1683432). Herein, this method was used to account for potential aberrant binding properties introduced by the click label. Details for the methods are provided in Examples 3 and 4.

To confirm the absence of unbound dye and to verify the concentration measured at the spectrometer, a size exclusion chromatography of the labeled antibodies was performed. Samples were separated using a BioSuite Diol (OH) column (Waters, 186002165) with a potassium dihydrogen phosphate buffer (pH 6.2) as the mobile phase at a flow rate of 0.5 ml/min. Detectors at 280 nm and 532 nm were used to quantify and analyze the labeled antibodies. The area under the curve (AUC) at 280 nm and 532 nm was extracted to calculate the concentration. The geo-mean of the AUC from all antibodies was computed and the deviation from each antibody to this geo-mean was identified. For an antibody to be reliable within the assay according to the invention, the difference from the geo-mean was expected to be below 15%.

1. A method for determining non-specific clearance of an antibody comprising the following steps:
a) incubating the antibody, which is conjugated to a pH-sensitive fluorescent dye, with primary human endothelial cells, and
b) determining the fluorescence intensity of the primary human endothelial cells of step a),
whereby non-specific clearance of the antibody is detected if the fluorescence intensity of the primary human endothelial cells determined in step b) is higher than the fluorescence intensity of the primary human endothelial cells determined in the absence of the antibody.

2. The method according to claim 1 further comprising the following step:
c) determining the fluorescence intensity of the primary human endothelial cells not incubated with/in the absence of the antibody.

3. The method according to any one of claims 1 to 2, wherein the primary human endothelial cells are washed prior to the determination of the fluorescence intensity.

4. The method according to any one of claims 1 to 3, wherein the dye has a fluorescence intensity change between a physiological pH of about 7 and an acidic pH in the range of pH 4 to 5 of about 10-fold determined at the same concentration of the dye and with the same excitation wavelength.

5. The method according to any one of claims 1 to 4, wherein the dye is pHAb of Formula I.

6. The method according to any one of claims 1 to 5, wherein the dye is conjugated to the antibody at residue 297 (numbering according to Kabat).

7. The method according to any one of claims 1 to 6, wherein the dye is conjugated to the antibody via a sulfo DBCO-PEG4-amine linker of Formula II.

8. The method according to any one of claims 1 to 7, wherein the dye is conjugated to a linker and the linker is conjugated to the antibody and has a structure of Formula III.

9. The method according to any one of claims 1 to 8, wherein the fluorescence intensity is determined by FACS by determining the shift of the fluorescence maximum.

10. The method according to any one of claims 1 to 9, wherein the fluorescence intensity is the geometric mean fluorescence intensity determined by FACS.

11. The method according to any one of claims 1 to 10, wherein the primary human endothelial cells are primary human liver endothelial cells.

12. The method according to any one of claims 1 to 11, wherein the incubating is for up to 4 hours.

13. The method according to any one of claims 1 to 12, wherein the incubating is at least for 0.5 hours.

14. The method according to any one of claims 1 to 13, wherein the antibody has an Fc-region of the human IgG1 or IgG4 subclass.

15. The method according to any one of claims 1 to 14, wherein the antibody is a bispecific antibody.


SEQUENCE LISTING (5 sequences)

SEQ ID NO: 1 - protein, 441 aa, synthetic construct
  REGION 1..441: note = anti-pTau antibody HC
EVQVLESGGGLVQPGGSLRLSCAVSGFSLSSNAINWVRQAPGKGLEWVGYIAVSGNTYYASWAKGRFTISRDNSKNTVYLQMNSLRAEDTAVYYCGKSNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 218 aa, synthetic construct
  REGION 1..218: note = anti-pTau antibody LC
AQVMTQSPSSLSASVGDRVTITCRSSQSVRTNRLAWFQQKPGQPPKRLIYSASTLDYGVPSRFSGSGSGTEFTLTISSLQPEDFATYYCLGYFDSSADIVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, synthetic construct
  REGION 1..449: note = anti-Her3 antibody HC
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 220 aa, synthetic construct
  REGION 1..220: note = anti-Her3 antibody LC
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK